(19) United States
(12) Patent Application Publication
(10) Pub. No.: US 2014/0314686 A1
(43) Pub. Date: Oct. 23, 2014

(54) POLYPHENOL/FLAVONOID COMPOSITIONS AND METHODS OF FORMULATING ORAL HYGIENIC PRODUCTS

(71) Applicant: API Genesis, LLC, Fairfax, VA (US)

(72) Inventors: Philip J. Birbara, West Hartford, CT (US); Jeffrey Cummings, Weston, MA (US)

(21) Appl. No.: 14/215,984

(22) Filed: Mar. 17, 2014

Related U.S. Application Data
(60) Provisional application No. 61/791,384, filed on Mar. 15, 2013.

(51) Int. Cl. A61K 8/49; A61K 8/66; A61Q 11/00; A61K 8/22; A61K 8/19; A61K 8/21; A61K 8/37

(52) U.S. Cl. CPC A61K 8/498; A61K 8/21; A61K 8/66; A61K 8/37; A61K 8/4973; A61K 8/22; A61K 8/19; A61Q 11/00
USPC 424/48; 424/49; 424/52; 424/50; 424/55; 424/53

(57) ABSTRACT

Microemulsions and soluble alkali metal salts of relatively insoluble aglycone polyphenols within oral hygienic products are disclosed for treating oral inflammatory disorders. The formulations can act as a bactericide or bacteriostat. The methods include the process associated with the formation of a high temperature polyphenol/surfactant concentrate, a nano-particulate precipitation process in the presence of a surfactant and the solubilization of relatively insoluble aglycone polyphenols/flavonoids by the formation of soluble alkali metal salts within alkaline oral compositions.

This application is based on and claims the benefit of priority to U.S. App. No. 61/791,384 filed on Mar. 15, 2013, the entire contents of which is expressly incorporated by reference herein.

### FIELD OF THE INVENTION

The present invention provides oral compositions for treating various oral inflammatory conditions, such as gingivitis and periodontitis.

The orally acceptable compositions can be present in various different forms such as a dentifrice, paste, gel, powder, mouth rinse, mouthwash, tooth hardener, medication, anti-calculus composition, film, slurry, injectable solution, and lozenge.

More particularly, compositions and methods for improving the aqueous solubility of aglycone polyphenols are disclosed. Such compositions and methods utilize stable polyphenol concentrates, microemulsions, and alkali polyphenol salts. Also included are methods for inhibiting and/or stopping bacterial growth.

### BACKGROUND OF THE INVENTION

Individual flavonoids can vary greatly in their biological activity (or be inactive), both in terms of toxicity and effectiveness against microbes such as viruses and bacteria. Compositions comprising polyphenolic compounds have been reported to have a wide range of biological activities, such as anti-oxidant, anti-inflammatory, anti-bacterial and anti-viral activities.

Polyphenols, and in particular the relatively insoluble aglycone forms of the flavone and flavonol flavonoids, have anti-cariogenic properties. Several in vitro and in vivo studies have investigated the effects of these flavonoids against bacterial microorganisms including Streptococcus mutans and inflammatory infections.

All flavonoids, a subset of polyphenol compounds, have the same basic chemical structure, a three-ringed molecule with hydroxyl (OH) groups attached. Flavonoids have the following general molecular structure as noted below

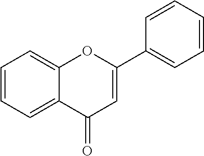 [SMILES: O=c1cc(-c2ccccc2)oc2ccccc12]

Flavonoids comprise approximately 5,000 naturally occurring compounds. A multitude of other substitutions can occur, giving rise to the many types of flavonoids, including the flavones (e.g., apigenin, luteolin, and so forth), flavonols (e.g., quercetin, myricetin, and so forth), flavonones (e.g., narigenin, hesperidin, and so forth), flavonols (or catechins) (e.g., epicatechin, gallocatechin, and so forth), anthocyanidins (e.g., cyaniding, pelargonidin, and so forth), and isoflavones (e.g., gunistein, daidezin, and so forth). Studies have demonstrated that flavones possess anti-oxidant, anti-mutagenic, anti-carcinogenic, anti-inflammatory, anti-proliferative, and anti-progression properties. (Patel, D, et al., Apigenin and cancer chemoprevention: Progress, potential, and promise, Intl. J. Oncology 2007 January; 30(1): 233-45).

Prior studies by researchers at the University of Rochester Medical Center noted that aglycone flavonoids, in particular the flavone and flavonol components within propolis, a resinous bee product, were responsible for inhibiting the growth of oral microorganisms and associated activity of the enzyme glucosyltransferase (GTF). Several compounds, mainly polyphenols, have been identified in this natural product. Thirty compounds, including flavonoids, cinnamic acid derivatives, and terpenoids found in propolis, were tested for the ability to inhibit GTFs B, C, and D from Streptococcus mutans and GTF from S. sanguinis (GTF Ss).

The researchers noted that flavones and flavonols were potent inhibitors of GTF activity in solution. Apigenin, a 4′,5,7-trihydroxyflavone, was the most effective inhibitor of GTFs; both in solution (90.5 to 95% inhibition at a concentration of 135 microg/ml) and on the surface of sHA beads (30 to 60% at 135 μg/ml) and was also cited as a novel and most potent natural inhibitor of MT activity, (Koo et. al., Effects of Compounds Found in Propolis on Streptococcus Mutans Growth and on Glucosyltransferase Activity, Antimicrob Agents Chemother, 2002 May; 46(5): 1302-9.)

U.S. Patent Application 2004/0057908 teaches an oral composition which includes an organoleptically suitable carrier and an amount of a terpenoid and a flavonoid, dispersed in the carrier, which is effective to prevent or treat dental caries, dental plaque formation, gingivitis, candidiasis, dental stomatitis, aphthous ulceration, or fungal infection. The invention also relates to various uses of oral compositions, containing a terpenoid, a flavonoid, or both, such uses include: inhibiting the activity of surface-bound glusosyltransferase; treating or inhibiting dental caries, gingivitis, candidiasis, and denture stomatitis; inhibiting the accumulation of microorganisms on an oral surface; and/or treating or inhibiting aphthous ulcerations on an oral surface.

Nearly all apigenin studies related to cancer and other research studies, including the cited studies of Koo et al, have utilized dimethyl sulfoxide (DMSO) and 100% ethyl alcohol as the solvent of choice due to the poor solubility of apigenin in water (0.003) milligram per milliliter (mg/ml)) as well as other aqueous and organic solvents suitable for oral compositions. (Li et al, Evaluation of Apigenin and [G-³H], Apigenin and analytical method development, J. of Pharmaceutical Sciences. Vol. 86, No. 6, June 1997). However, the use of apigenin vehicles containing DMSO and/or 100% ethyl alcohol is not suitable for human oral formulations.

Apigenin has been shown to be antifungal making it effective for treating denture stomatitis (Herrera et al., The Antifungal effect of 6 commercial extracts of Chilean propolis on Candida spp. Cien. Inv. Agr. 37 (1): 75-84 2010.) Also, it can act as an anti-inflammatory so it has also been investigated for periodontal disease Anti-inflammatory effects of apigenin on nicotine and lipopolysaccharide stimulated human periodontal ligament cells via heme oygenase. November 2009 1374-1380 Vol. 9 Issue 12 Int Immunology). Apigenin is a strong antioxidant and was shown to inhibit oral carcinogenesis in hamsters. (S. Sylvan, Chemotherapeutic potential of apigenin in 7,12, dimethyl anthracene induced experimental oral carcinogenesis, Eur J pharmacol 2011 November 670 23)

Apigenin possesses anti-inflammatory activity in human periodontal ligament (hPDL) cells and works through a novel mechanism involving the action of heme oxygenase-1 (HO-1)1. Thus, apigenin has benefits as a host modulatory agent in the prevention and treatment of periodontal disease associated with smoking and dental plaque. (Gil-Saeng Jeong et al; Anti-inflammatory effects of apigenin on nicotine-and lipopolysaccharide-stimulated human periodontal ligament cells via heme oxygenase-1, International Immunopharmacology, Vol.: 9, November 2009).

U.S. Patent Application 2012/0213842 teaches methods of making and using flavonoids.

U.S. Pat. No. 8,637,569 relates to methods of increasing the solubility of poorly soluble compounds and methods of making and using formulations of such compounds. U.S.

U.S. Ser. No. 61/886,977 teaches beverages containing polyphenols and methods of making same.

As background, hydrogen peroxide, in combination with and/or sodium bicarbonate, can in a combination decompose rapidly. Peroxides, such as hydrogen peroxide, typically can break down in the presence of alkalinity, heat, light and/or metal ions as follows:

2H₂O₂→2H₂O+O₂ (gas)

In addition, sodium carbonate peroxide breaks down into sodium carbonate and hydrogen peroxide as follows:

2Na₂CO₃.3H₂O₂ (Sodium Carbonate peroxide)→2Na₂CO₃+3H₂O₂

The hydrogen peroxide acts as a potent oxidizing agent, and as an anti-microbial agent. Further the high alkalinity of sodium carbonate (˜pH=10.5) boosts the oxidizing effect of hydrogen peroxide.

Similarly, sodium bicarbonate can break down in the presence of hydrogen peroxide, heat and/or water as follows:

2NaHCO₃→Na₂CO₃+H₂O+CO₂ (gas)

Several clinical trials have demonstrated that sodium bicarbonate dentifrices have enhanced plaque removal effectiveness of tooth brushing to a significantly greater extent than the non-sodium bicarbonate dentifrice products. (Putt, M. S. et al, Enhancement of Plaque Removal Efficacy by Tooth Brushing with Baking Soda Dentifrices: Results of Five Clinical Studies, J. Clin Dent, 2008, 19(4); 11-9.) (Mankodi, S. et al, Evaluation of the Effects of Brushing on the Removal of Dental Plaque, J. Clin Dent, 1998; 9(3) 57-60.) (Drake, D. R., Enhanced Bactericidal Activity of Arm and Hammer Dental Care, Am J Dent, 1995, December; 8(6): 308-312.). Further the topical application of the combination of hydrogen peroxide and sodium bicarbonate exhibited synergistic oxidative antimicrobial activity. (Miyasaki, K. T. et al, Antimicrobial Properties of Hydrogen Peroxide and Sodium Bicarbonate individually and in Combination Against Selected Oral, Gran-negative, Facultative Bacteria, J Dent Res, 1986, September 85 (9), 1142-1148)

The composition of several dentifrices containing significant concentrations of sodium bicarbonate and peroxide is considered to be noteworthy because of the difficulty of combining peroxide and baking soda in a way that avoids rapid decomposition due to interaction of the peroxide with the sodium bicarbonate when in solution. Several commercially available (e.g. Arm & Hammer PeroxiCare®) formulations use a combination of peroxide and sodium bicarbonate with slightly more than a 1% water concentration. The commercially available formulations utilize combinations of PEG-8 and a PEG/PPG 116/66 copolymer for stabilizing the peroxide and sodium bicarbonate ingredients.

Thus, there is a need for creating oral formulations and methods containing stable aglycone flavonoid concentrates, microemulsions and alkali aglycone flavonoid salts for treating oral inflammatory disorders and inhibiting bacterial growth in mammals.

Due to the inadequacy of mechanical removal of plaque via tooth brushing, there is much interest in chemical inhibition of plaque formation.

### OBJECTS OF THE INVENTION

It is an object of this invention to provide improved oral compositions containing polyphenols including aglycone flavones, flavonols, and flavanols. Such compositions are toothpastes, mouthwashes, mouth rinses, gum, candy, as well as in toothache, sore throat, and cold sore medications.

It is another object to provide improved oral compositions containing a combination of aglycone flavones, aglycone flavonols, aglycone flavanols and other relatively insoluble polyphenol ingredients.

It is another object of this invention to provide stable formulations containing soluble alkali metal polyphenol salts, e.g. flavone salts with the oxidative sodium bicarbonate and sodium carbonate peroxide combination.

Other objects will become apparent to those skilled in the art upon a further reading of the specification.

### SUMMARY OF THE INVENTION

The present invention relates to polyphenol containing compositions for use in the preparation of oral compositions, such as toothpastes, mouthwashes or mouth rinses, gums, and candies. In particular, it relates to oral compositions comprising antibacterial, antioxidant, anti-inflammatory polyphenol/flavonoid formulations, their preparation and use. The invention discloses oral products containing therapeutically effective concentrations of aglycone flavonoid compounds as antiplaque and anti-inflammatory agents within suitable oral vehicles or carriers (“orally acceptable”—not harmful to the patient when used in the mouth) for treating oral inflammatory disorders and inhibiting and/or killing bacteria. The amounts used of the polyphenol are “therapeutically effective” i.e. amounts needed as part of the composition to obtain the desired result such as reducing the symptoms of gingivitis, periodontitis, inhibiting the activity of glucosyltransferase, etc. in mammals including humans.

The present invention, in various embodiments, provides oral compositions for treating and/or inhibiting various oral inflammatory conditions, such as gingivitis, periodontitis, etc. The oral compositions can be present in various different forms. For example, the oral compositions can be at least one of a dentifrice, paste, gel, powder, mouth rinse, mouthwash, tooth hardener, oral film, anti-calculus composition, film, slurry, injectable solution, and lozenge.

Microemulsions of relatively insoluble aglycone polyphenols are disclosed to improve the aqueous solubility within these oral hygienic products. The methods of production include both the formation of both a high temperature surfactant/polyphenol concentrate, and a nano-particulate precipitation process in the presence of a sufficient surfactant concentration.

Soluble flavone salt formulations within alkaline oral compositions containing sodium bicarbonate, sodium carbonate peroxide and a peroxide stabilizer have been experimentally determined to be effective for treating, oral inflammatory disorders and inhibiting bacterial growth.

The alkaline soluble polyphenol salt containing toothpaste compositions of the present invention can include solid inorganic peroxide which will allow the release of nascent oxygen upon brushing of the teeth with the composition such that the nascent oxygen is activated and released upon contact with the saliva in the mouth or the addition of water. Further, a water soluble or water emulsifiable coating encapsulates the peroxide ingredient.

The invention provides stable formulations containing soluble alkali metal flavone salts with the oxidative sodium bicarbonate and sodium carbonate peroxide combination.

The invention relates to a composition comprising, consisting of, or consisting essentially of:

i) a polyphenol, and
ii) an orally acceptable carrier,
wherein said polyphenol is in the form of an alkali metal salt or a concentrate.

Typical polyphenols include flavonoids and stilbenes.

Typical carriers are water (e.g. deionized), glycerin, ethanol, sorbitol, and propylene glycol. Additives are included depending on the form of the composition, e.g. s toothpaste, mouth wash, and mucoadhesive vehicle.

The compositions inhibit the activity of surface bound glucosyltransferase, and inhibit or destroy microorganisms (particularly those producing glucosyltransferase) upon administration to an oral cavity.

In an advantageous embodiment, the composition does not include DMSO, and/or the composition does not include greater that 40% ethanol. Typically the polyphenol is greater than 0.01-5 percent by weight of the composition. The composition generally is in the form of a liquid, a gel, a paste, a spray, a powder, a gum, a lozenge or a tablet. In an advantageous embodiment, the composition, e.g. toothpaste, comprises: a flavonoid, sodium bicarbonate, and a peroxide, e.g. sodium carbonate peroxide, and optionally, a polymer for stabilizing the sodium bicarbonate and/or peroxide.

The invention also relates to a method of inhibiting the activity of surface bound glucosyltransferase, and inhibiting the activity of soluble and surface-bound microorganisms responsible for dental caries comprising: administering to the oral cavity a therapeutically effective amount of a composition of the invention.

In another embodiment, the invention relates to a method of providing therapeutically effective levels of a polyphenol in an oral cavity comprising administering to the oral cavity a composition of the invention.

In a still further embodiment, the invention relates to a method of delivering a polyphenol systemically to a mammal comprising administering buccally to the oral cavity of a mammal a composition of the invention, typically at least once weekly, or advantageously once a day.

In another embodiment, the invention relates to a method for treating an oral disease or condition in a mammal comprising: administering to the oral cavity of said mammal a sustained therapeutically effective amount of a composition of the invention. Administering to the oral cavity comprises administering to one or more of a tooth, a mucosal surface, a tongue surface, a surface on complete or partial dentures, or a combination thereof. The composition is administered at least once daily. Alternatively, the composition in the form of a rinse is administered to the oral cavity for a period of about 30-60 seconds. In the form of a paste or gel, the composition is administered to the oral cavity for a period of at least 1 minute. Another embodiment of the invention is dental floss coated with a composition of the invention.

In a further embodiment, the invention relates to a method of making a polyphenol containing toothpaste or oral rinse composition comprising:
a) mixing a heat stable polyphenol compound with a heat stable nonionic surfactant [(e.g. s. PS80 and Polyoxyl-40-hydrogenated castor oil (Cremophor RH40)] to form a mixture,
b) heating said mixture resulting from step a) to a temperature such that said heat stable polyphenol compound is solubilized to form a concentrate,
c) cooling said concentrate resulting from step b),
d) adding the solubilized polyphenol concentrate of step c) to a toothpaste or oral rinse to form said polyphenol containing toothpaste or oral rinse composition.

In another embodiment, the invention relates to a method of making a polyphenol containing toothpaste or oral rinse composition comprising:
a) mixing a polyphenol with a toothpaste or oral rinse formulation to form a composition,
b) adding an alkali metal hydroxide to the composition of step a) to a pH level of about 10 to form an alkali metal polyphenol salt within said toothpaste or oral rinse formulation, and
c) acidifying the product of step (b) with an acidic agent to form said polyphenol containing toothpaste or oral rinse composition.

The alkali metal hydroxide is typically sodium hydroxide or potassium hydroxide or a mixture thereof, and the acidifying agent is citric acid, acetic acid, ascorbic acid, hydrochloric acid or a mixture thereof.

### DETAILED DESCRIPTION OF THE INVENTION

The subject invention relates to compositions comprising an antibacterial, antioxidant, anti-inflammatory polyphenol/flavonoid compositions, their preparation and use. It is desirable to incorporate flavones particularly apigenin, luteolin, quercitin, etc., as part of oral formulations to aid in the maintenance of proper oral hygiene. The methods associated with preparation of polyphenol/flavonoid containing compositions are useful in the preparation of oral compositions, such as toothpastes, mouthwashes or mouth rinses, gums, drinks and candies.

The oral compositions are useful for treating and/or inhibiting various oral inflammatory conditions, such as gingivitis, periodontitis, etc. The compositions are well suited to inhibit the buildup of microorganisms that promote gingivitis, dental caries and the development of the dental plaque structure. Plaque is a haven for oral microorganisms and continues to build up in the oral cavity until it can mineralize to form calculus (also known as tartar) as well as cause plaque associated gum disease. The microorganisms that form the biofilm are mainly Streptococcus mutants and anaerobes, with the composition varying by patient age and location in the mouth.

Toothpaste and oral rinses are an essential part of oral hygiene, and help to prevent gum disease. Gum disease, or gingivitis, occurs when there is an abundance of bacteria and subsequent plaque present around teeth. Untreated cases can eventually lead to tooth loss, so it is important to exercise daily preventive measures that include toothpaste for the treatment and prevention of gum disease.

Gingivitis (gum inflammation) usually precedes periodontitis (gum disease). However, not all gingivitis progresses to periodontitis. If left untreated, gingivitis may progress to periodontitis which can be a major cause of tooth loss in adults.

Microemulsions of relatively insoluble aglycone polyphenols are disclosed to improve their aqueous solubility within oral hygienic compositions.

The methods of making the compositions include i) the formation of a high temperature polyphenol/surfactant concentrates, ii) a nano-particulate precipitation process in the presence of a surfactant, and iii) the solubilization of relatively insoluble aglycone polyphenols/flavonoids within alkaline oral compositions and iv) combinations thereof.

The invention includes methods for increasing the solubility of poorly soluble aglycone flavonoid compounds with surfactants such as polysorbate, polyoxyl hydrogenated castor oil, etc. in formulations. As previously noted, many aglycone flavonoids and specifically apigenin are poorly soluble in aqueous solutions thus severely limiting their bioavailability for oral, pharmaceutical and neutraceutical applications.

### Compositions of the Invention

The polyphenol compositions of the invention can be present in various forms. For example, the compositions can be in the form of a dentifrice (toothpaste), paste, gel, powder, liquid, mouthwash, mouth rinse, cream, lotion, tooth hardener, oral film, anti-calculus composition, film, slurry, injectable solution, gum, lozenge, tablet, candy, food or beverage. The compositions can take the form of a solution (e.g., mouthwash), a suspension, or an emulsion. These compositions include a suitable carrier for the aglycone flavonoid/polyphenol ingredients.

The compositions of this invention are compositions of polyphenols. The polyphenols include flavonoids, stilbenes, curcumins, and lignans. As used herein, “poorly soluble” or “relatively aqueous insoluble” are polyphenols or flavonoids having a solubility in water of less than 1 mg/ml, and particularly less than 0.1 mg/ml.

It has been found that improved solubility and stability of the aqueous aglycone flavonoid microemulsions can be greatly improved by adjusting the pH by the addition of pH-controlling agents, buffers, to maintain pH within a range of from 5.5 to 11.0.

### Flavonoids

Flavonoids of the invention include flavones, flavonols, flavanols, proanthocyanidins, dihydroflavonols, flavones, and derivatives thereof. Exemplary are aglycone flavonoids without limitations and include apigenin, luteolin, chrysin, quercetin, hesperitin, naringin, genistein, daidzein, epigallocatechin gallate, catechin and combinations thereof.

The chemical structures of some commonly occurring plant flavonoids are listed in Table I.

TABLE I
CHEMICAL, STRUCTURES OF SOME COMMONLY OCCURING PLANT FLAVONOIDS

| Structure | Represtative flavonoids |
|---|---|
| Flavones 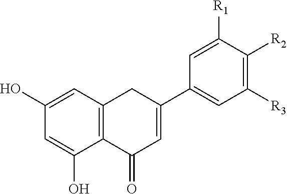 | R1 = H, R2 = OH: Apigenin R1 = R2 = OH: Lutcolin |
| Flavonols 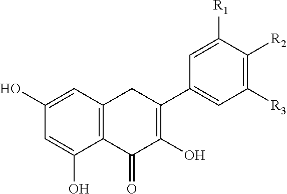 | R2 = OH, R1 = R3 = H: Kaempferol R1 = R2 = OH, R3 = H: Quereetin R1 = R2 = R3 = OH: Myrieetin |
| Isoflavones 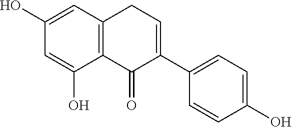 [SMILES: O=C1C(c2ccc(O)cc2)=CCc2cc(O)cc(O)c21] | R1 = H: Daidzein R1 = OH: Genistein |
| Flavanols 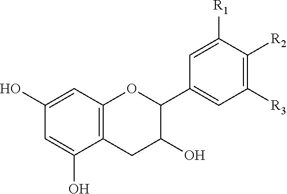 | R1 = R2 = OH, R3 = H: Catechins R1 = R2 = R3 = OH: Gallocatechin |
| Flavanones 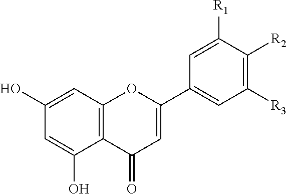 | R1 = H, R2 = OH: Naringenia R1 = R2 = OH: Enodicyrol R1 = OH, R2 = OCH3: Hespereum |

Apigenin is a member of the flavone structural class and is chemically known as 4′,5,7,-trihydroxyflavone. Apigenin has the following structural formula:

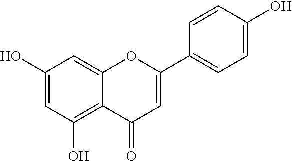 [SMILES: O=c1cc(-c2ccc(O)cc2)oc2cc(O)cc(O)c12]

Luteolin is also a member of the flavone structural class and is chemically known as 3′,4′,5,7-tetrahydroxyflavone. Luteolin has the following structural formula:

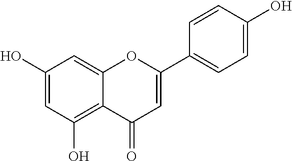 [SMILES: O=c1cc(-c2ccc(O)cc2)oc2cc(O)cc(O)c12]

Epigallocatechin gallate (EGCG) is a type of catechin that is a most abundant in tea and is a potent antioxidant that may have therapeutic application in the treatment of many disorders. EGCG has been found to effective in the treatment of Sjogrens syndrome. EGCG has the following structural formula:

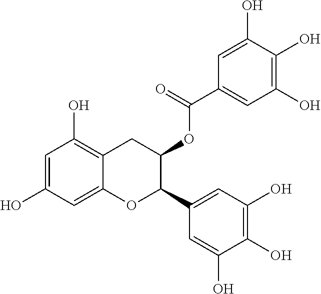 [SMILES: O=C(O[C@@H]1Cc2c(O)cc(O)cc2O[C@@H]1c1cc(O)c(O)c(O)c1)c1cc(O)c(O)c(O)c1]

The aglycone flavonoid is present in the formulations of the invention in amounts to inhibit the activity of soluble or surface-bound bacterial microorganisms so as to prevent or to treat dental caries, dental plaque formation, gingivitis, periodontitis, candidiasis, dental stomatitis, and fungal infections.

An effective amount of aglycone flavonoids present in the oral compositions of the invention are greater than 0.01 wt. %, greater than or 0.1 wt. %. Typically, the aglycone flavonoid is present in an amount that is between 0.1 to about 5 wt. %, or from about 0.3 to about 2.0 wt. %, or from 0.5 wt. % to about 3 wt. %.

### Carriers and Additives

The particular choice of carrier will depend, at least in part, upon the desired form of the oral composition: for example a toothpaste or gel, a powder, a solution (e.g., mouthwash or mouth rinse), a suspension, an emulsion, a lozenge, a mucoadhesive vehicle, a beverage a tablet, a capsule or a gum. Conventional ingredients that can be used to form the carriers listed above are well known to the skilled artisan. Any suitable orally acceptable vehicle can be used, such as those described in U.S. Pat. No. 4,894,220 hereby incorporated by reference in its entirety.

Preferably, such carrier materials are selected for compatibility and stability with all of the constituents of the formulation including the active ingredient(s), such as aglycone flavonoid(s) and the optional one or more oral care active agent compounds selected for the oral composition. Further, as described previously above, the carrier ingredient can also serve as a bioavailability-enhancing agent, either as an efficacy-enhancing agent or a solubilizing agent for the active ingredients.

As recognized by one of skill in the art, the oral compositions optionally include other materials in addition to those components previously described, including for example without limitation, a cariostatic agent, a humectant, an abrasive agent, a gelling agent, a flavoring agent, a desensitizing agent, an anti-calculus agent, a whitening agent, a surfactant, a binding agent, a preservative, a buffering agent, an opacifying agent, a coloring agent, and combinations thereof. It is understood that while general attributes of each of the above categories of materials may differ, there may be some common attributes and any given material can serve multiple purposes within two or more of such categories of materials.

Water is typically an element of the oral compositions. Water employed in the preparation of commercially suitable toothpastes should preferably be deionized and free of organic impurities. The amounts of water include the free water which is added plus that which is introduced with other materials. In certain embodiments, the oral compositions are anhydrous; e.g. s., stannous fluoride and calcium sodium phosphosilicate formulations. In another embodiment, the amount of water is less than 5 wt. % (e.g., PeroxiCare® type formulation).

The oral product can also be a liquid, such as a mouthwash or mouth rinse which typically contains an aqueous non-toxic lower aliphatic alcohol, advantageously having about 2-30 wt. % by weight of a non-toxic alcohol, such as ethanol, n-propanol, or isopropanol, with water, and often about 5-35 percent of humectant.

Cariostatic agents (non-flavonoid cariostatic agents) can be provided in each form of the oral composition. Fluoride in various forms is the most popular active ingredient in toothpaste to prevent cavities. The additional fluoride in toothpaste has beneficial effects on the formation of dental enamel and bones. Suitable cariostatic agents include sodium fluoride, stannous fluoride, aminefluoride, sodium monofluorophosphate, sodium trimeta-phosphate, triclosan, casein, or combinations thereof. If desired, the cariostatic agent can be present in an amount between about 0.01 to about 2 weight percent, more typically between about 0.02 to about 1 weight percent.

Humectants can also be employed in the oral compositions, particularly toothpastes and gels and oral rinses. These agents are used to give toothpaste texture, prevent drying out by retaining moisture and prevent hardening of the paste on exposure to air. Suitable humectants include, glycerin, propylene glycol, polyethylene glycol, xylitol, sorbitol, maltitol, lactitol, or the like. The humectant can also be used as the bulk carrier, in which case it can be present in an amount of about 5 to about 90 weight percent, more typically about 10 to about 60 weight percent.

Abrasive agents are typically employed in dentifrice compositions. Abrasives constitute at least 50 wt. % of typical toothpaste. These insoluble particles help remove plaque from the teeth. The removal of plaque and calculus helps minimize cavities and periodontal disease. Suitable abrasive agents include silica gel, zirconosilicate, silicic anhydride, aluminosilicate, calcium carbonate, calcium pyrophosphate, aluminum oxide, aluminum hydroxide, calcium hydrogen phosphate dihydrate or anhydride, aluminum silicate, insoluble sodium metaphosphate, magnesium carbonate, calcium sulfate, and combinations thereof. Sodium bicarbonate is a particularly effective abrasive agent that also provides a mild teeth-whitening action. It neutralizes acidic saliva, thus maintaining an alkaline environment in the mouth, even hours after brushing. An alkaline environment is not favorable for and hampers the formation of dental plaque. It is a natural teeth whitener and hence effective to remove stains. It is an effective teeth-cleaning agent and due to its abrasive action it can clear off those brown and yellow stains. While brushing, baking soda infiltrates the tooth's enamel, which helps to reduce the appearance of the stains that are on the surface of the teeth. Abrasives can generally be employed in effective amounts of between about 20 to about 90 weight percent, more typically about 20 to about 60 weight percent.

Gelling agents or thickeners can be used in the various compositions. Suitable gelling agents include carrageenan, sodium carboxymethyl cellulose, alkali metal alginates such as sodium alginate, gums, polyvinyl alcohol, and vee gum or the like. Typically, the gelling agents are employed in amount of about 0.3 to about 5 weight percent.

Flavorants in toothpaste comes in a variety of colors and flavors intended to encourage use of the product. Three most common flavorants are peppermint, spearmint, and wintergreen. The respective oils, e.g. peppermint oil, provide these flavors. More exotic flavors include anise, apricot, bubblegum, cinnamon, fennel, lavender, ginger, vanilla, lemon, orange, and pine. Unflavored toothpastes exist.

Desensitizing agents can be introduced in some of the oral composition to alleviate sensitivity of individuals whose teeth are sensitive to thermal shock, chemicals, etc. Suitable desensitizing agents include potassium nitrate, potassium citrate, potassium chloride, potassium tartrate, potassium bicarbonate, potassium oxalate, and strontium salts. Desensitizing agents can be present, either individually or collectively, in an amount of about 0.1 to about 5 weight percent, more typically about 0.1 to about 3 weight percent.

Anti-calculus agents can be introduced to the oral composition to treat tartar formation. Suitable anti-calculus agents include alkali-metal pyrophosphates, hypophosphite-containing polymers, organic phosphonates, phosphocitrates, zinc salts and combinations thereof. Anti-calculus agents can be present, either individually or collectively, in an amount of about 0.1 to about 5 weight percent, more typically about 0.1 to about 3 weight percent.

Whitening agents can be employed in some forms of the oral composition. Some of these toothpastes contain peroxide, the same ingredient found in tooth bleaching gels Suitable whitening agent including sodium carbonate peroxide, calcium peroxide, sodium tripolyphosphate and hydrogen peroxide. Whitening agents can be employed in amounts of about 0.5 to about 5 weight percent.

Surfactants can also be employed in the various oral compositions. The purpose of these agents is to facilitate the distribution of the paste in the mouth by lowering the surface tension and helping to loosen plaque and other debris from the tooth surface. They also contribute to the foaming action of toothpastes. Fluorides work better in combination with detergents, which help the remineralization process of tooth enamel. Any of a variety of types of surfactants can be utilized, including anionic, nonionic, cationic and zwitterionic or amphoteric surfactants, or combinations thereof. Exemplary anionic surfactants include, without limitation, sodium lauryl sulfate, sodium lauryl sarcosinate, a-olefin sulfate, tabulate, lauryl monoglyceride sulfate, lauryl monoglyceride sulfonate, and combinations thereof. Exemplary nonionic surfactants include, without limitation, TWEEN, lauroyl diethanol amide, stearyl monoglyceride, sucrose fatty acid esters, lactose fatty acid esters, lactitol fatty acid esters, maltitol fatty acid esters, polyoxyethylene sorbitan monostearate, and combinations thereof. Exemplary ampholytic surfactants include, without limitation, betain and amino acid type surfactants. Surfactants can be present in amount of about 0.5 to about 15 weight percent, more typically about 0.5 to about 10 weight percent.

Binding agents maintain the consistency of toothpaste, tablet or lozenges. It binds all the ingredients in the formulation together. Hydrocolloids, such as alginate or xanthan, are often used as binding agents. Other binding agents include sodium carboxymethyl-cellulose, gum arabic as well as synthetic polymers such as polyacrylates and carboxyvinyl polymers. Binders can be present in amounts of about 0.5 to about 50 weight percent depending on the form of the oral composition.

Preservatives play an important role in keeping the oral compositions free from microorganisms. Sodium benzoate is a commonly used preservation agent that prevents the buildup of microorganisms in oral products and also functions to provide a degree of cariostatic activity. Other commonly used preservatives in oral compositions include, methyl paraben, and ethyl paraben.

Buffering agents useful in the present compositions are those that are capable of maintaining the desired pH thereby promoting its stability and desired properties. The pHs of oral compositions are generally in the range of about 4.5 to about 11, or about 6.5 to about 9.0. The pH can be adjusted with the addition of acidic ingredients such as citric acid or benzoic acid or alkaline ingredients such as sodium or potassium hydroxide and buffered to maintain pH with salts such as sodium citrate, benzoate, carbonate, or bicarbonate, disodium hydrogen phosphate, sodium dihydrogen phosphate, etc.

Opacifying agents can also be added to various oral compositions of the present invention. Titanium dioxide is a white powder that adds opacity to the compositions. Titanium dioxide can be present in an amount of about 0.25 to about 5 weight percent.

Coloring agents provide toothpaste with pleasing colors. Artificial dyes are used to make red, green, and blue toothpastes. Coloring agents can be present in an amount of about 0.01 to about 5 weight percent.

Other ingredients such as minerals, vitamins, herbs, CoQ10, propolis, echinacea etc., are often added in the formulation of toothpaste, to make it more effective in controlling bad breath and plaque formation. Vitamins include Vitamins C (L-ascorbic acid) and D, thiamine, riboflavin, calcium pantothenate, niacin, folic acid, nicotinamide, pyridoxine, cyanocobalamin, para-aminobenzoic acid, bioflavonoids, and mixtures thereof.

### Polyphenol/Surfactant Concentrate Formulations

The polyphenol/surfactant “concentrates”, see commonly owned U.S. Pat. No. 8,637,569 hereby incorporated by reference in its entirety, can be utilized to a variety of oral compositions including toothpaste or gel, a powder, a solution (e.g., mouthwash or mouth rinse), a suspension, an emulsion, a lozenge, a tablet or a gum.

### Polyphenol Microemulsion Formulations

The polyphenol/surfactant microemulsions, see commonly owned U.S. Patent Application 2012/0213842 hereby incorporated by reference in its entirety, can be utilized to a variety of oral compositions including toothpaste or gel, a powder, a solution (e.g., mouthwash or mouth rinse), a suspension, an emulsion, a lozenge, a tablet or a gum.

### Polyphenol Salt Formulations

Advantageous embodiments of the invention are stable formulations containing aqueous soluble alkali metal polyphenol, e.g. flavone salts. The soluble alkali flavone ingredients, advantageously the sodium salt of apigenin, within the compositions, e.g. dentifrice compositions, comprise 0.01 to 5 wt. %, 0.1 wt. % to 5 wt. %, or from 0.3 wt. % to 3 wt. %.

The dentifrice compositions of the present invention including alkali metal salts of a polyphenol such as a flavone, typically contain a limited water concentration, i.e. from 1 wt. % to 5 wt. % and more advantageously, from 2 wt. % to 3 wt. %.

Particularly advantageous embodiments of the invention are stable formulations containing soluble alkali metal polyphenol salts, e.g. flavone salts with the oxidative sodium bicarbonate and peroxide, e.g. sodium carbonate peroxide combination.

Suitable peroxides in the composition include encapsulated solid inorganic peroxides advantageously alkali metal carbonate peroxides such as sodium carbonate peroxide which will allow the release of nascent oxygen upon brushing of the teeth with the composition. The nascent oxygen is generated and released upon formulation contact with the saliva in the mouth or the addition of water. Sodium carbonate peroxide concentrations comprise from about 2 wt. % to about 10 wt. % and advantageously from 1 wt. % to about 10 wt. %.

The bicarbonate salt ingredients, advantageously sodium bicarbonate, of the dentifrice compositions comprise about 10 wt. % to about 60 wt. % of the composition and advantageously from about 20 wt. % to about 50 wt. %.

The stabilizing material is included in the composition of the invention in an amount effective so as to inhibit breakdown of the peroxide, the soluble aglycone flavonoid salt and/or sodium bicarbonate in the composition during storage in a closed container, but at a concentration sufficient so as to allow release of nascent oxygen from the peroxide when the composition is in contacted with saliva during brushing of teeth. Suitable stabilizing ingredients include polymer compositions include PEG and PEG/PPG copolymers. Ascorbic acid in may be added to the formulation to stabilize alkali metal aglycone flavonoid salts. The stabilizing ingredients are included in the composition in an amount of from 1 wt. % to about 20 wt. % and advantageously from about 2 wt. % to about 10 wt. %.

### Methods of Preparing Formulations of the Invention

### (a) The Formation of Polyphenol/Surfactant Concentrate Formulations

Heating relatively aqueous insoluble polyphenol compounds to temperatures approaching their melting points with a heat stable nonionic surfactants to elevated temperatures (typically >100° C.), not exceeding the boiling point or decomposition point of either the active agent (e.g. polyphenol) or the heat stable solubilizing agent (e.g. surfactant), and then cooling said mixture, results in a “concentrate.” See U.S. Pat. No. 8,637,569.

This process can enhance dissolution, and achieve a significantly higher concentration of the polyphenolic compound in solution with the surfactant. Furthermore, the resulting solution “concentrate” is not supersaturated; such that said polyphenol/surfactant concentrate can then be used to subsequently prepare desired formulations. The molar ratio of active agent (polyphenol) to solubilizing agent (surfactant) is typically 1:2 to 1:5, and at times much greater, e.g. 1:2 to 1:20 depending on the active agent/surfactant combination. Upon cooling to room temperature, the concentrates are not supersaturated solutions even though the concentrations of the compounds are greater than their saturation concentration at ambient conditions-room temperature (temperature below that necessary to overcome the intermolecular self-association forces). The concentrate is stable and the compounds (or active agents) stay in solution at ambient temperatures for periods of time (weeks, months, advantageously 1 or 2 years) sufficient for making formulations from the concentrates.

Apigenin/Polysorbate 80 (PS80) formulations can be made as follows:
Apigenin powder and PS80 are mixed in the ratio from about 5 to 10 wt. % of apigenin to 95 to 90 wt. % PS80. This mixture is thoroughly stirred to form a paste-like blend.
The mixture is then slowly heated to relatively high temperatures to temperatures approaching 300 degree C.
A dark brown transparent liquid results such that all the solid apigenin is solubilized in the PS80 mixture.
Upon cooling to ambient temperatures, a clear viscous brown liquid results.
Based on a 5.0 wt. % concentration of apigenin in the PS80 solvent, a stable apigenin concentrate containing is 50 mg/ml is formed
It was unanticipated that high temperature levels were necessary to cause the high solubility level of apigenin and other relatively water insoluble aglycone flavonoids.

The invention includes the use of heat stable surfactants such as the polysorbate and Polyoxyl-40-hydrogenated castor oil (Cremophor RH40) surfactants to achieve elevated soluble concentrates of the other aglycone flavonoids including the flavones apigenin and luteolin, the flavonol quercetin, the flavanone hesperitin and the polyphenol resveratrol. (See Example 1)

The polyphenol/surfactant concentrates can be added to a variety of carriers and additives to form a toothpaste or gel, a powder, a solution (e.g., mouthwash or mouth rinse), a suspension, an emulsion, a lozenge, a tablet or a gum.

### (b) The Formation of a Flavonoid/Polyphenol Microemulsion Formulations

To prepare microemulsions of the subject invention, the teachings of commonly owned U.S. Patent Application 2012/0213842 is incorporated by reference in its entirety.

The teachings of this inventive method are applicable to preparing microemulsions of poorly soluble flavonoids/polyphenols having solubility in water less than 1 mg/ml, and particularly less than 0.1 mg/ml.

The “nano-particulate” is well suited for the addition of relatively aqueous insoluble flavonoids/polyphenols with aqueous oral compositions including mouth rinses and mouthwashes.

In one embodiment, a flavonoid/polyphenol microemulsion can be made as follows:
The mixing of a flavonoid/polyphenol with an alkali metal component (e.g., alkali metal hydroxide(s) and/or alkaline metal salt(s)) to form an alkali metal flavonoid/polyphenol salt within an aqueous solution.
Adding a surfactant in the ratio from about 5 to 20 wt. % of the flavonoid/polyphenol to 95 to 80 wt. % of a surfactant; preferable a nonionic surfactant.
This mixture is thoroughly stirred to form a uniform clear solution.
Adjusting (e.g., acidifying) the alkali metal flavonoid/polyphenol salt with an agent (e.g., an acidic agent such as acetic acid and/or hydrochloric acid) to a pH level required to form a clear and stable microemulsion.

Stable microemulsions with apigenin and the nonionic surfactants, Polysorbate 80 and Cremophor RH40, were achieved provided a pH level of 8.0 to 8.5 was maintained. For luteolin, it as determined that a pH of 7 was required to maintain a stable aqueous microemulsion; and for resveratrol, a pH of >4.5 to 7.5.

The solubilizing agent(s) (surfactant(s)) can be present in various amounts in the oral composition, such as an amount sufficient to dissolve the mixture of flavonoids/polyphenols and to prevent precipitation thereof upon dilution with saliva. The solubilizing agent(s) can also be present in an amount effective to increase the uptake of the antibacterial agent and the mixture of flavonoids/polyphenols by dental tissue. The solubilizing agent(s) are advantageously present at about 0.01 wt. % to 10% wt. %; and most advantageously, between 0.05 wt. % to 2 wt. %.

Generally toothpaste can be said to have pHs ranging between 5.5 and 11. For example fluoride will form fluoric acid and lower the pH, while baking soda or similar will increase the pH. However, many toothpaste formulations are mildly alkaline with pH ranging from 7-10 depending on its additives. The alkaline pH of toothpaste helps neutralize the plaque acids that cause tooth decay.

It was discovered that improved solubility and stability of the aqueous aglycone flavonoid (e.g. s flavone and flavonol) microemulsions can be greatly improved by adjusting the pH by the addition of pH-controlling agents from about 5.5 to 11, or from 6.5 to 9.0.

In the oral cavity, bacterial metabolism releases organic acids that attack the dental enamel. Brushing of the teeth with an alkaline toothpaste will neutralize organic acids and also serves as a cleansing agent. Thus, teeth stay cleaner and avoid more damage with toothpaste. In general, the ratio of the suspended and dispersed micro-particulate form of apigenin to the dissolved alkali salt form within the vehicle is increased as the pH level of the formulation is reduced from the slightly basic (pH of approximately 8 (e.g., pH of 7 to 9) to the moderately acidic (pH of approximately 4 (e.g., pH of 3.5 to 5)).

The degree of acidity and alkalinity (pH) can have a dramatic impact on the color of selected aglycone flavonoids and can provide a qualitative colorimetric method for the determination of the presence of aglycone flavonoids. For example, the addition of alkali metal hydroxides such as sodium hydroxide to slightly acidic solutions of many aglycone flavonoids result in the formation of colored aglycone flavonoid salts. Examples of color changes due to the formation of alkali metal salts includes the yellow colored flavone and deep red/orange catechin salts.

### (c) The Formation of Soluble Polyphenol Salt Formulations

As shown in the Examples below, flavone salt formulations of the invention can be formed by adding a flavone to a composition having a preexisting high pH, or alternatively mixing a flavone into a composition having a lower pH and then increasing the pH of the composition by adding an alkali metal hydroxide such as sodium hydroxide to the composition to 7.5 to 11.

It was experimentally determined that the toothpaste formulations containing both sodium bicarbonate and sodium carbonate peroxide together with stabilizing polymer formulations had pHs of ˜10-10.5. When aglycone flavone (i.e., apigenin and luteolin) powders were added to dentifrices including sodium bicarbonate, sodium bicarbonate peroxide and a peroxide stabilizer, it was also determined that soluble flavone sodium salts were formed that were stable in these highly oxidative peroxide compositions. The presence of stable soluble alkali metal flavone salts with highly anti-oxidant and anti-inflammatory properties in an emulsion formulation was totally unanticipated since aglycone flavones are prone to oxidative decomposition.

### Uses of the Compositions of the Invention

The present invention provides oral compositions for treating or preventing dental diseases or conditions including dental plaque, dental caries, periodontal disease, oral cancer, oral chemotherapy sequelae, gingivitis, herpetic lesion, cold sore, apthous ulcer, toothache, wound, tooth sensitivity, denture stomatitis, fungal, viral or bacterial infection, and various oral inflammatory conditions.

The oral compositions of the present invention are also well suited to inhibit the accumulation of microorganisms which promote dental caries, gingivitis, candidiasis, denture stomatitis, or formation of dental plaques. Treating mammals by using the oral compositions of the present invention slows or stops the accumulation of microorganisms, such as Streptococci mutans.

Such oral preparations are typically applied by contacting natural or artificial teeth and gums through brushing with a dentifrice or toothpaste, or by contacting teeth and gums by rinsing the oral cavity for about 15-90 seconds, or in the case where a lozenge, candy or chewing gum are used by sucking or chewing in the oral cavity, or in the case of a mouthspray by spraying the oral surfaces at least once weekly, or advantageously daily.

Example 11 below shows that apigenin formulated into products for oral health, including toothpastes, gums, and lozenges (and presumably other products such as mouthwashes) can increase apigenin concentrations within saliva in an acute manner with retention seen after 1 hour and possibly much longer (as observed in the chronic user of PeroxiCare® with apigenin). Finally, this Example supports the clinical finding demonstrated in Example 10.

The anti-inflammatory, antimicrobial and anti-oxidant properties of flavonoids provide the ability to successfully treat gingivitis and periodontal disease ailments. Indeed the subject formulations enable the delivery of solubilized polyphenols including aglycone flavonoid ingredients at concentrations not achievable by currently practiced methods. Together with other desirable toothpaste formulation ingredients, polyphenols including apigenin, and optionally therapeutic fluorides, protect against plaque, gingivitis, cavities and tooth sensitivity. Together they deliver a unique, comprehensive protection to teeth.

The following Examples are illustrative, but not limiting of the compositions and methods of the invention. Other suitable modifications and adaptations of a variety of conditions and parameters normally encountered which are obvious to those skilled in the art are within the spirit and scope of the invention.

### EXAMPLES

### Example 1

### Preparation of the Apigenin/Polysorbate 80 (PS80) Concentrate

### Required Ingredients Includes:

9.25 grams of a highly purified PS80
0.75 grams of Apigenin powder

### Procedure:

1. Add 9.25 grams of the highly purified PS80 to a 50 cc “Pyrex” beaker.
2. Add 0.75 grams of Apigenin powder to the PS80.
3. Heat the PS80/Apigenin mixture to a temperature slightly in excess of ˜275° C. At about 200° C., it will be observed that the mixture will take on a light brown/reddish color which will darken when the Apigenin is completely solubilized at ˜275° C.
4. The Apigenin/PS80 solution is set aside and allowed to cool to <100° C.

### Example 2

### Apigenin Containing Toothpaste Formulation

### Arm & Hammer Dental Care Advance White Breath Freshening Baking Soda Toothpaste, Frosted Mint

### Active Ingredients: Sodium Fluoride (0.24%) (Anticavity Toothpaste)

### Inactive Ingredients: Water, Baking Soda (Sodium Bicarbonate), Sorbitol, Hydrated Silica, Glycerin, Tetrasodium Pyrophosphate, Flavor, Sodium Saccharin, Cellulose Gum, Sodium Lauroyl Sarcosinate, Sodium Lauryl Sulfate, Titanium Dioxide.

TABLE II
The Composition of an Apigenin Containing
Arm & Hammer Advance White Toothpaste

| | QUANTITY | COMPOSITION |
|---|---|---|
| INGREDIENTS | (grams) | (wt. %) |
| Arm & Hammer Baking | 92.0 | 92.0 |
| Soda Advance White | | |
| Toothpaste | | |
| H₂O Added | 3.4 | 3.4 |
| Polysorbate 80⁽²⁾ | 3.2 | 3.2 |
| Solubilized Apigenin⁽¹⁾⁽⁴⁾ | 0.2 | 0.2 |
| Apigenin Powder⁽³⁾ | 1.2 | 1.2 |
| TOTAL | 100 | 100 |

Note:
⁽¹⁾The “Apigenin Solubilized” ingredient refers to the methodology for preparing Apigenin/PS80 concentrates.
⁽²⁾Super Refined PS80 obtained from Croda Inc.
⁽³⁾The “Apigenin Powder”, 98⁺% apigenin, Skyherb Technologies LTD, is partially solubilized in the alkaline toothpaste formulation.
⁽⁴⁾The apigenin content per gram of toothpaste is >25 times that of the Koo formulations required to inhibit the glucosyltransferase enzyme.

100 grams of the Arm & Hammer Apigenin Containing formulation was prepared as follows:
1. 92.0 grams of the Arm & Hammer white colored toothpaste was added to a 300 ml Pyrex glass beaker. The pH of the toothpaste was initially determined via pH indicator strips to be decidedly alkaline at approximately >9.5 but slightly <10.
2. 3.4 grams of a previously prepared Polysorbate/Apigenin concentrate containing 0.2 grams of Apigenin dissolved in 3.2 grams of PS80 was added to Step 1. The combined mixture was thoroughly stirred until a uniform light yellow blend was observed.
3. 3.4 gram of distilled water was added to the mixture from Step 2 and the resulting mixture thoroughly stirred to obtain a uniform blend
4. 1.2 grams of apigenin powder was added to the mixture from Step 3 and stirred until a uniform bled was obtained. The resulting mixture was a distinct yellow color—indicative of the solubilizing of apigenin as a sodium salt.

### Example 3

### Luteolin Containing Toothpaste Formulation

### Colgate Whitening Tartar Control Plus a Whitening Fluoride Toothpaste, Crisp Mint

### Active Ingredients: Sodium Fluoride (0.24%) (0.15% w/v Fluoride Ion)

Inactive Ingredients: Sorbitol, Water, Hydrated Silica, Glycerin, PEG 12, Pentasodium Triphosphate, Tetrasodium Pyrophosphate, Sodium Lauryl Sulfate, Flavor, Sodium Hydroxide. Sodium Saccharin, Cellulose Gum, Carrageenan (Red Seaweed), Titanium Dioxide.

TABLE III
The Composition of a Luteolin Containing “Colgate
Tartar Protection” Toothpaste

| | QUANTITY | COMPOSITION |
|---|---|---|
| INGREDIENTS | (grams) | (wt. %) |
| Colgate Tarter Protection | 92.0 | 92.0 |
| Whitening Toothpaste | | |
| H₂O Added | 3.4 | 3.4 |
| Polysorbate 80⁽²⁾ | 3.2 | 3.2 |
| Solubilized Luteolin ⁽¹⁾ | 0.2 | 0.2 |
| Luteolin Powder ⁽³⁾ | 1.2 | 1.2 |
| TOTAL | 100 | 100 |

Note:
⁽¹⁾ The “Solubilized Luteolin” ingredient refers to the methodology for preparing Luteolin/PS80 concentrates.
⁽²⁾Super Refined PS80 obtained from Croda Inc.
⁽³⁾ The “Luteolin Powder”, 98⁺% luteolin, Skyherb Technologies LTD, is partially solubilized in the alkaline toothpaste formulation.

100 grams of the whitening Luteolin Containing formulation was prepared as follows:
1. 92.0 grams of the Colgate whitening toothpaste was added to a 300 ml Pyrex glass beaker. The pH of the toothpaste was initially determined via pH indicator strips to be decidedly alkaline at approximately >9.5 but slightly <10.
2. 3.4 grams of a previously prepared Polysorbate 80/Luteolin concentrate containing 0.2 grams of Luteolin dissolved in 3.2 grams of PS80 was added to Step 1. The combined mixture was thoroughly stirred until a uniform light yellow blend was observed.
3. 3.4 gram of distilled was added to the mixture from Step 2 and the resulting mixture thoroughly stirred to obtain a uniform blend
4. 1.2 grams of luteolin powder was added to the mixture from Step 3 and stirred until a uniform bled was obtained. The resulting mixture was a distinct yellow color—indicative of the solubilizing of luteolin as a sodium salt.

### Example 4

### Resveratrol Containing Whitening Toothpaste Formulation

### Crest Complete Multi-Benefit Toothpaste, Whitening Plus Scope, Minty Fresh Stripe Toothpaste Formulation

### Active Ingredients: Sodium Fluoride (0.243%) (0.15% w/v Fluoride Ion) (Anticavity Toothpaste)

Inactive Ingredients: Sorbitol, Water, Hydrated Silica, Disodium Pyrophosphate, Sodium Lauryl Sulfate, Flavor, Sodium Hydroxide, Alcohol (0.7%), Xanthan Gum, Sodium Saccharin, Glycerin, Carbomer 956, Cellulose Gum, Polysorbate 80, Sodium Benzoate, Cetyl Pyridinium Chloride, Benzoic Acid, Titanium Dioxide, Blue 1 Lake (CI-42090), Yellow 5 Lake.

TABLE IV
The Composition of a Resveratrol Containing Crest Complete
Multi-Benefit, Whitening Plus Scope Toothpaste

| | QUANTITY | COMPOSITION |
|---|---|---|
| INGREDIENTS | (grams) | (wt. %) |
| Crest Complete Multi-Benefit, | 98.6 | 98.6 |
| Whitening Plus Scope | | |
| Toothpaste Formulation | | |
| NaOH Crystals | 0.1 | 0.1 |
| Resveratrol Powder ⁽¹⁾⁽²⁾ | 1.2 | 1.2 |
| Citric Acid Crystals | 0.1 | 0.1 |
| TOTAL | 100 | 100 |

Note:
⁽¹⁾ The “Resveratrol Powder”, 98⁺% resveratrol, Pure Bulk Inc., is completely solubilized in the alkaline toothpaste formulation.
⁽²⁾Resveratrol was solubilized as a sodium salt and/or nano-emulsion within the toothpaste formulation.

100 grams of the formulation was prepared as follows:
1. 98.6 grams of the toothpaste was added to a 300 ml Pyrex glass beaker. The pH of the Crest toothpaste was initially determined via pH indicator strips to be decidedly alkaline at approximately 8.5.
2. About 0.1 grams of fine NaOH crystals were added to the toothpaste and thoroughly mixed into the toothpaste. A uniformly light green blend resulted with a measured pH of ˜10. The elevated alkalinity of the blended mixture resulted in the solubilizing of resveratrol as its sodium salt.
3. The pH the mixture from Step 2 was adjusted to a pH 8.5 by the addition of about 0.1 grams of citric acid crystals.

### Example 5

### Resveratrol Containing Mouthwash Formulation

### Active Ingredients

Thymol (0.064%), Eucalyptol (0.092%), Methyl Salicylate (0.060%), Menthol (0.042%)

### Inactive Ingredients Water, Alcohol (26.9%), Benzoic Acid, Poloxamer 407, Sodium Benzoate, Caramel

The antiseptic mouthwash formulation LISTERINE® rapidly penetrates the biofilm to kill plaque and gingivitis germs.

TABLE V
The Composition of a Resveratrol Containing
LISTERINE ORIGINAL MOUTHRINSE

| | QUANTITY | COMPOSITION |
|---|---|---|
| INGREDIENTS | (grams) | (wt. %) |
| Listerine ® Original A | 98.4 | 98.4 |
| Mouthrinse Formulation | | |
| NaOH Crystals | 0.2 | 0.2 |
| Resveratrol Powder ⁽¹⁾⁽²⁾ | 1.2 | 1.2 |
| Citric Acid Crystals | 0.2 | 0.2 |
| TOTAL | 100 | 100 |

Note:
⁽¹⁾ The “Resveratrol Powder”, 98⁺% resveratrol, Pure Bulk Inc., is completely solubilized in an alkaline mouthrinse formulation.
⁽²⁾Resveratrol was solubilized as a sodium salt and/or nano-emulsion within the mouthrinse formulation.

100 grams of the Listerine® Resveratrol Containing Mouth Rinse formulation was prepared as follows:
1. 98.4 grams of the Listerine® caramel colored mouth rinse was added to a 300 ml Pyrex glass beaker. The pH of the Listerine® caramel colored mouth rinse was initially determined via pH indicator strips to be decidedly acidic at approximately 4.5.
2. 1.2 grams of Resveratrol powder was added to the solution of Step 1.
3. About 0.2 grams of fine NaOH crystals were added to the mixture from Step 2 and thoroughly mixed. A uniformly caramel colored blend resulted with a measured pH of ˜10. The elevated alkalinity of the blended mixture resulted in the solubilizing of resveratrol as its sodium salt.
4. The pH the mixture from Step 3 was adjusted to a pH 6.5 by the addition of about 0.2 grams of citric acid crystals. A transparent solution resulted with a resveratrol concentration of 1.2 mg/ml.

### Example 6

### Luteolin Containing Mouthwash Formulation

### Active Ingredients

Thymol 0.064%, Eucalyptol 0.092%, Methyl Salicylate 0.060%, Menthol 0.042%

### Inactive Ingredients

Water, Alcohol (26.9%), Benzoic Acid, Poloxamer 407, Sodium Benzoate, Caramel LISTERINE® Antiseptic mouth rinse rapidly penetrates the biofilm to kill plaque and gingivitis germs.

TABLE VI
The Composition of a Luteolin Containing
“LISTERINE ® ORIGINAL” MOUTH RINSE

| | QUANTITY | COMPOSITION |
|---|---|---|
| INGREDIENTS | (grams) | (wt. %) |
| Listerine ® Original Mouthrinse | 98.3 | 98.3 |
| NaOH Crystals | 0.1 | 0.1 |
| Luteolin Powder ⁽¹⁾⁽²⁾ | 1.5 | 1.5 |
| Citric Acid Crystals | 0.1 | 0.1 |
| TOTAL | 100 | 100 |

Note:
⁽¹⁾ The “Luteolin Powder”, 98⁺% luteolin, Skyherb Technologies LTD, is solubilized in the alkaline mouthrinse formulation
⁽²⁾Luteolin was solubilized as a sodium salt

1. 98.6 grams of the Listerine® caramel colored mouthrinse was added to a 300 ml Pyrex glass beaker. The pH of the Listerine® caramel colored mouthrinse was initially determined via pH indicator strips to be decidedly acidic at approximately 4.5.
2. 1.2 grams of Luteolin powder was added to the solution of Step 1.
3. About 0.2 grams of fine NaOH crystals were added to the mixture from Step 2 and thoroughly mixed. A uniformly caramel colored blend resulted with a measured pH of ˜10. The elevated alkalinity of the blended mixture resulted in the solubilizing of luteolin as its sodium salt.
4. The pH the mixture from Step 2 was adjusted to a pH 8 by the addition of about 0.2 grams of citric acid crystals. A transparent solution resulted with a luteolin concentration of 1.5 mg/ml.

### Example 7

### An Apigenin Containing Chewing Gum Formulation

Ingredients: Sorbitol, Gun Base, Glycerol, Natural and Artificial flavors; Less than 2% of: Hydrogenated Starch Hydrolysate, S, Lecithin, Aspartame-Acesulfame, Mannitol, Citric Acid, Aspartame, Malic Acid, Sucralose, Acesulfame K, Colors (Yellow 5 Lake, Blue 1 Lake), BHT (To maintain freshness).

TABLE VII
The Composition of an Apigenin Containing
Extra Spearmint Chewing Gum

| | QUANTITY | COMPOSITION |
|---|---|---|
| INGREDIENTS | (grams) | (wt. %) |
| 2 Sticks of Extra Spearmint | 5.93 | 95.95 |
| Gum | | |
| Polysorbate 80⁽¹⁾ | 0.2375 | 3.84 |
| Solubilized Apigenin ⁽²⁾⁽³⁾ | 0.0125 | 0.21 |
| TOTAL | 6.18 | 100.00 |

Note:
⁽¹⁾The “Apigenin Solubilized” ingredient refers to the methodology for preparing Apigenin/PS80 concentrates; (See Example 1).
⁽²⁾ Super Refined PS80 obtained from Croda Inc.
⁽³⁾The “Apigenin Powder”, 98⁺% Apigenin, Skyherb Technologies LTD, is partially solubilized in the alkaline toothpaste formulation.

6.18 grams of the Extra Spearmint apigenin containing formulation was prepared as follows:
1. 0.25 grams of a previously prepared Polysorbate 80/Apigenin liquid concentrate (See Example 1) containing 0.0125 grams of apigenin dissolved in 0.2375 grams of PS80 was added to 2 sticks of the Extra Spearmint Chewing Gum weighing 5.93 grams.
2. The Polysorbate 80/Apigenin liquid concentrate was readily and easily kneaded into the gum. The gum's physical structure and chewing quality was not noticeably altered. Significantly, the original light green/yellow color of the formulation was not visibly altered by the addition of the PS80/Apigenin concentrate.

The total quantity of dissolved apigenin computes to ˜12 mg for two sticks of gum which far exceeded the literature quantity of apigenin required to inhibit the activity of surface-bound glucosyltransferase to hinder microbial glucan-forming activity. In addition, the solubilized apigenin contributes to dental health via its anti-inflammatory and anti-oxidant properties. Apigenin's role inhibiting and/or stopping the accumulation of microorganisms contributes to the prevention of dental plaque, dental caries, gingivitis and other oral problems. Further the nonionic PS80 surfactant may further serve to aid in apigenin contact with the surface structure of the plaque.

The gums composition of the present invention can be combined with effective amounts of other components, such as other aglycone flavonoids that can contribute to prevent or treat dental plaque, dental caries, gingivitis and other dental problems.

### Example 8

### Formation of a Soluble Flavone Salt Formulations within an Alkaline Toothpaste Comprised of Sodium Bicarbonate, Peroxide and a Peroxide Stabilizer

Arm & Hammer PeroxiCare® Toothpaste Formulation

### Active Ingredient: Sodium Fluoride (0.24%)

### Inactive Ingredients: Sodium Bicarbonate (Baking Soda), PEG-8, PEG/PEG-116/66 Copolymer, Tetrasodium Pyrophosphate, Sodium Carbonate Peroxide, Silica, Sodium Saccharin, Flavor, Water, Sodium Lauryl Sulfate, Sodium Lauryl Sarcosinate.

TABLE VIII
The Composition of Alkaline Soluble Sodium
Apigenin Salt Containing Toothpaste

| | QUANTITY | COMPOSITION |
|---|---|---|
| INGREDIENTS | (grams) | (wt. %) |
| PeroxiCare ® Toothpaste | 150 | 99.6 |
| Formulation | | |
| Solubilized Apigenin Powder ⁽¹⁾⁽²⁾ | 0.6 | 0.4 |
| TOTAL | 150.6 | 100.00 |

Note:
⁽¹⁾ The Solubilized Apigenin ingredient refers to the formation of an alkaline sodium apigenin salt formed when apigenin added to the PeroxiCare ® toothpaste formulation.
⁽²⁾The “Apigenin Powder”, 98+% apigenin, Skyherb Technologies LTD, is partially solubilized in the alkaline toothpaste formulation.

BASIS: 100 cc of PeroxiCare® Toothpaste

1. Dispense 100 cc of PeroxiCare® toothpaste into a clean 200 ml Pyrex glass beaker which has been cleaned with an ethyl alcohol rinse.
2. Verify that the weight of the 100 cc of the PeroxiCare® toothpaste from Step 1 is 150 grams. If not, add or remove PeroxiCare® to achieve a weight of 150 grams.
3. Confirm that the pH of the PeroxiCare® from Step 2 is ≧10. The pH is determined by taking about <0.1 grams of the toothpaste formulation PeroxiCare® and thoroughly mixing it with ˜20 cc of distilled water contained within a 30 ml Pyrex glass beaker. A drop of this liquid mixture is then added to a strip of pH paper to determine the pH. Allow at least a minute of contact with the pH before comparing the color change to determine the pH. In the unlikely event that the pH is <10, the addition of sodium hydroxide crystals to the toothpaste formulation can be made to achieve a pH level ˜10.
4. Add 600 mg of apigenin powder to the mixture from Step 2 and thoroughly mix with a spatula for at 5 minutes to obtain a uniform yellow mixture.
5. The mixture from Step 4 containing 6 mg/ml of a sodium apigenin salt within a PeroxiCare® mixture is then poured into suitable airless dispensing tubes.

### Example 9

### The Treatment of Oral Inflammatory and Microbacterial Disorders Resulting from the Application of a Soluble Sodium Apigenin Salt Formulations within an Alkaline Toothpaste Comprised of Sodium Bicarbonate, Peroxide and a Peroxide Stabilizer

Ten patients had gingivitis diagnosed by bleeding on probing and gingival index. These patients used the toothpaste of Example 8 without flossing, rinse or professional care for one week. Each patient demonstrated significant improvement with little or no bleeding on probing and a healthier gingival index. The product successfully treated gingivitis for this patient population.

### Example 10

### A Double Blind, Randomized Trial Comparing Efficacy of an Alkaline with Apigenin Dentifrice for the Treatment of Gingivitis

### Primary Objective:

The primary objective of this study was to evaluate efficacy of alkaline apigenin toothpaste similar to that described in Example 8 for topical treatment of gingivitis.

### Overall Study Design:

This study was a randomized, double blind trial clinical comparing alkaline toothpaste with apigenin for the treatment of gingivitis. Twenty subjects were enrolled with varying severity of gingivitis, if not early periodontitis. Each subject had gingiva around at least 4 teeth with an index of 2, moderate inflammation, redness, edema and bleeding as described by Loe and Silness. Recorded clinical parameters included the following: (1) Gingival Index; (2) Bleeding Index (number of surfaces bleeding and severity by number of teeth present); (3) Periodontal Pocket Depths, and (4) Plaque Index.

A screening and examination visit took place at the study center where intraoral photographs of the selected subjects were taken. Ten subjects used the alkaline PeroxiCare® toothpaste, and 10 subjects used the alkaline toothpaste with apigenin exclusively for 2 weeks with no rinsing or flossing. Selected subjects were instructed to use only the test material provided for 2 minutes twice a day (morning and before bed) and given instruction in sulcular brushing technique and told to use a two-minute timer. Each subject was also shown the appropriate amount of test material to be put on the new brush that they were given. Each subject was instructed to brush each quadrant of the mouth for 30 seconds making a total brushing cycle of 2 minutes. Subjects were also instructed to keep a daily diary to further document their experience with the test material

The clinical parameters of Gingival Index, Bleeding Index, Periodontal Pocket Depth and Plaque Index were recorded at day1 and day15. Intraoral photographs of teeth involved were taken on both days and the diaries were reviewed in order to collect all necessary information.

### Toothpaste Preparation:

Toothpastes used in this study were prepared by the procedures described in Example 8. Food grade dyes were utilized so that the 2 formulations had the same color so neither the investigator nor patient knew which formulation they received. The 2 formulations were labeled B and C. The formulator who prepared the B and C products was the only person who will knew the compositions of the formulations as noted in the Table IX.

TABLE IX
Toothpaste Sample Compositions

| | ⁽¹⁾Alkaline | | | |
|---|---|---|---|---|
| Sample | Toothpaste | Apigenin | TOTAL | |
| Identification | (Wt. %) | (Wt. %) | (Wt. %) | pH |
| B Alkaline | 100 | 0 | 100 | ~10.2 |
| Toothpaste | | | | |
| C Alkaline | 99.3 | 0.7 | 100 | ~10.2 |
| Toothpaste | | (10 mg/ml) | | |
| with Apigenin | | | | |

Note:
⁽¹⁾The Alkaline Toothpaste ingredients are listed in Example 8

### Study Evaluations

### Gingival Index (0-3 Point Scale):

As described by Loe and Silness:
Score 0—Normal;
Score 1—Mild inflammation, slight color change, no bleeding;
Score 2—Moderate inflammation, edema redness bleeding on probing;
Score 3—Severe inflammation redness, edema, easy bleeding.

The final gingival index score equals summation of the scores per surface/total number of surfaces (4 per tooth). These scores were then added for the four teeth involved in the study.

### Bleeding Index: (0-5) Point Scale):

An early sign of gingivitis is bleeding on probing and, in 1971; Muhlemann and Son described the Sulcus Bleeding Index (SBI). The criteria for scoring are as follows:
Score 0—health looking papillary and marginal gingiva no bleeding on probing;
Score 1—healthy looking gingiva, bleeding on probing;
Score 2—bleeding on probing, change in color, no edema;
Score 3—bleeding on probing, change in color, slight edema;
Score 4—bleeding on probing, change in color, obvious edema;
Score 5—spontaneous bleeding, change in color, marked edema

Ten patients had gingivitis diagnosed by bleeding on probing and gingival index. These patients used the toothpaste of Example 8 without flossing, rinse or professional care for one week. Each patient demonstrated significant improvement with little or no bleeding on probing and a healthier gingival index. The product successfully treated gingivitis for this patient population for one week.

### Periodontal Pocket Depth:

Depth measurements in mm were taken with a periodontal probe at 6 points around each tooth. These scores were added for the four teeth chosen in the study.

### Results

Both the alkaline toothpaste and alkaline toothpaste with apigenin formulations resulted in substantial improvements for the 2 week period study duration for all measured scores as noted in Table X. Significantly, the alkaline toothpaste with apigenin resulted in improvements of 3.1%, 34.2% and 30.6% of the Pocket Depth, Bleeding and Gingival Indexes, respectively when compared to the alkaline toothpaste formulation without apigenin.

The anti-inflammatory and antimicrobial properties of apigenin contributed to the improvements of the oral health noted in this study.

Some subjects noted that the addition of apigenin to the alkaline toothpaste improved the taste and flavor of the formulation.

TABLE X
A Comparison of the ⁽¹⁾Alkaline Toothpaste and Alkaline
Toothpaste with Apigenin Improvements (% Improvements
after Morning & Evening Brushing Teeth for 2 Weeks)

| | POCKET | BLEEDING | GINGIVAL |
|---|---|---|---|
| SAMPLE | DEPTH (% | INDEX (% | INDEX (% |
| IDENTIFICATION | Improvement) | Improvement) | Improvement) |
| Alkaline | 10.31 | 21.82 | 10.76 |
| Toothpaste | | | |
| Alkaline | 10.63 | 29.82 | 14.05 |
| Toothpaste with | | | |
| Apigenin | | | |

Note:
⁽¹⁾The Alkaline Toothpaste ingredients are listed in Example 8

### Example 11

### Apigenin Content of Formulated Products and of Apigenin in Saliva and Sulcus Following Product Use

### Primary Objectives:

The primary objective of this study was to evaluate the retention of apigenin in the saliva and sulcus fluid when using toothpastes of the invention.

### Overall Study Design:

Six subjects provided saliva samples with 5 different apigenin containing toothpastes: (1) before brushing, (2) after brushing without rinsing, (3) immediately after rinsing, and (4) one hour after rinsing. Each subject brushed their teeth for 2 minutes using the sulcular brushing technique with ˜1.8 g toothpaste on a new toothbrush. One subject (chronic user) followed the brushing routine for twice daily, morning and night, for more sixty days prior to the start of the study.

Four (4) different sulcus locations were measured using absorbent paper points. The infiltrated paper points were collected after brushing and 1 hour post rinsing. All determinations of apigenin in saliva and sulcus fluid were conducted in a manner blind to the treatment assignments.

### Sample Preparations:

Toothpaste samples were prepared by the procedures described in the “Methods of Preparing Formulations of the Invention Section” and Examples 1, 3 & 8.

Details of the 5 toothpaste samples utilized in this study are provided in Table XI.

TABLE XI
Toothpaste Sample Characteristics

| | Apigenin | | | |
|---|---|---|---|---|
| | Content | Apigenin | | Number of |
| TOOTHPASTE | (mg/ml) | Form | pH | Subjects |
| PeroxiCare ® | 8 | Sodium | ~10.5 | 1 acute use |
| | | Salt | | 1 chronic use |
| ⁽²⁾Crest Pro- | 10 | Sodium | ~10.5 | 1 |
| Health | | Salt | | |
| Crest Pro-Health | 2 | PS80 | ~6.0 | 1 |
| | | Concentrate | | |
| Crest Pro-Health | 10 | Nano | ~6.0 | 1 |
| | | particulates | | |
| Tom's of Maine | ⁽¹⁾ N/A | — | ~8.0 | 1 |
| Propolis & Myrrh | | | | |

Note:
⁽¹⁾ Propolis presumably contains a low apigenin concentration
⁽²⁾Modified by the addition of NaOH

The ingredients contained within each of the toothpaste formulations are listed in Table XII.

TABLE XII
Toothpaste Sample ingredients

| | Active | |
|---|---|---|
| Toothpaste | Ingre- | |
| Type | dients | Inactive Ingredients |
| PeroxiCare ® | Sodium | Sodium Bicarbonate (Baking Soda), PEG-8, |
| | Fluoride | PEG/PEG-116/66 Copolymer, Tetrasodium |
| | (0.24%) | Pyrophosphate, Sodium Carbonate Peroxide, |
| | | Silica, Sodium Saccharin, Flavor, Water, |
| | | Sodium Lauryl Sulfate, Sodium Lauryl |
| | | Sarcosinate. |
| Crest Pro- | Stannous | Glycerin, Hydrated Silica, Sodium Hexa- |
| Health | Fluoride | metaphosphate, Propylene Glycol, PEG 6, |
| | (0.454%) | Water, Zinc Lactate, Flavor, Trisodium |
| | | Phosphate, Sodium Gluconate, Sodium |
| | | Lauryl Sulfate, Sodium Saccharin, Carra- |
| | | geenan, Stannous Chloride, Xanthan Gum, |
| | | Polyethylene, Titanium Dioxide, Blue 1 |
| | | Lake, Blue 1 |
| Tom's of | — | Calcium Carbonate, Water, Glycerin, |
| Maine | | Sodium Bicarbonate, Xylitol, Carrageenan, |
| Propolis & | | Fennel Oil, Sodium Lauryl Sulfate, Myrrh |
| Myrrh | | Resin Extract, Propolis Extract |

### Experimental Methods:

Apigenin in the toothpaste products was extracted using a liquid-liquid protocol. Briefly, 0.5 gm of toothpaste was mixed with 5 ml H₂O. After 2 minutes of rigorous vortexing, apigenin in the resulting mixture was extracted with 15 ml ethyl acetate. After 2 minutes of rigorous vortexing and 15 minutes of centrifugation, aliquots of the ethyl acetate and water fractions were dried under N₂ gas and reconstituted for apigenin analysis using HPLC-ECD. The concentration of apigenin in toothpaste products is the sum of the apigenin content in both water and ethyl acetate fractions.

Saliva was collected at each time point in 50 ml Falcon tubes and immediately transferred to a freezer. After collection, paper points infiltrated with sulcus fluid were also placed in 50 ml and stored in a freezer. All samples were transported to Tufts University for analysis within 8 hours of collection.

Apigenin in saliva and sulcus was quantified using a high performance liquid chromatograph (HPLC) with electrochemical detection (ECD). Apigenin in 1 ml saliva or 4 paper points containing sulcus fluid was extracted using 3 ml acetonitrile. After 2 minutes of rigorous vortexing, the mixture was spun at 2,000 rpm for 15 minutes at 4° C. The supernatant was dried under N₂ gas, reconstituted with mobile phase A, and analyzed for apigenin with HPLC-ECD. The limit of quantification (LOQ) for this assay is 15 ng/ml saliva.

The concentration of apigenin in saliva, paper points, and the toothpaste products was calculated using a standard curve constructed with authenticated apigenin. The standard curve was linear with R² value of 0.9973.

### Results

The analytical apigenin concentrations in the toothpaste, gum and lozenge test samples are itemized in the Table XIII.

TABLE XIII
The Analytical Determined Apigenin Content of the Toothpastes

| | | | | | Normalized |
|---|---|---|---|---|---|
| | Apigenin | | | Apigenin | Apigenin |
| TOOTH- | Content | Apigenin | | Content | Content to |
| PASTE | (mg/ml) | Form | pH | (μg/g) | 1 mg/ml |
| PeroxiCare ® | 8 | Sodium | ~10.5 | 5603 | 700.4 |
| | | Salt | | | |
| ⁽²⁾Crest | 10 | Sodium | ~10.5 | 7964 | 797.4 |
| Pro-Health | | Salt | | | |
| Crest | 2 | PS80 | ~6.0 | 1799 | 899.5 |
| Pro-Health | | Concen- | | | |
| | | trate | | | |
| Crest | 10 | Nano | ~6.0 | 7609 | 760.9 |
| Pro-Health | | Partic- | | | |
| | | ulates | | | |
| Tom's of | ⁽¹⁾ N/A | — | ~8.0 | 0.23 | 0.04 |
| Maine Propolis | | | | | |
| & Myrrh | | | | | |

Note:
⁽¹⁾ Propolis presumably contains a low apigenin concentration
⁽²⁾Modified by the addition of NaOH

The analytical measured apigenin content in saliva and sulcus fluid of toothpaste subjects as a function of time are listed in Table XIV.

TABLE XIV
The Analytical Determined Apigenin Content in the
Saliva and Sulcus Fluids of Toothpaste Subjects

| | | SALIVA⁽⁴⁾ | | | | SULCUS⁽⁵⁾ | |
|---|---|---|---|---|---|---|---|
| | | TIME POINTS | | | | | |
| | Apigenin | Apigenin Content ⁽¹⁾ | | | | | |
| | Content | (μg/ml) ⁽¹⁾ (μg) | | | | | |
| TREATMENT | (mg/ml) | 1 | 2 | 3 | 4 | 1 | 2 |
| Crest Pro- | 10 | ND⁽³⁾ | 679.76 | 8.1 | 0.09 | 0.41 | 0.17 |
| Health Nano- | | | (67.98) | (0.81) | (0.01) | (0.04) | (0.02) |
| Particulates | | | | | | | |
| PeroxiCare | 8 | 0.72 | 235.00 | 26.00 | 3.29 | 0.28 | 0.04 |
| Sodium | | (0.09) | (29.38) | (3.25) | (0.41) | (0.04) | (0.01) |
| Apigenin Salt | | | | | | | |
| (Chronic Use) | | | | | | | |
| ⁽⁶⁾Crest Pro- | 10 | ND⁽³⁾ | 459.00 | 8.72 | 3.97 | 0.80 | 0.19 |
| Health | | | (45.90) | (0.87) | (0.40) | (0.08) | (0.02) |
| Sodium | | | | | | | |
| Apigenin Salt | | | | | | | |
| PeroxiCare ® | 8 | ND⁽³⁾ | 119.00 | 37.40 | 0.96 | 0.21 | 0.07 |
| Sodium | | | (14.88) | (4.66) | (0.12) | (0.03) | (0.01) |
| Apigenin Salt | | | | | | | |
| (Acute Use) | | | | | | | |
| Crest Pro- | 2 | ND⁽³⁾ | 29.10 | 8.08 | 0.14 | 0.15 | 0.02 |
| Health | | | (14.05) | (4.04) | (0.07) | (0.08) | (0.01) |
| PS80/Apigenin | | | | | | | |
| Concentrate | | | | | | | |
| Tom's of Maine | N/A | ND⁽³⁾ | ND⁽³⁾ | ND⁽³⁾⁾ | ND⁽³⁾ | ND⁽²⁾ | ND⁽²⁾ |
| Propolis & | | | | | | | |
| Myrrh | | | | | | | |

Note:
⁽¹⁾ Values in Parenthesis normalized to 1 mg/ml apigenin in the toothpaste formulations.
⁽²⁾Apigenin in the sulcus fluid cannot be expressed as μg/ml because the fluid volume in the paper points is not known.
⁽³⁾ND, Not Detected.
⁽⁴⁾Saliva Time Points: (1) before brushing, (2) after brushing without rinsing, (3) immediately after rinsing, and (4) one hour after rinsing.
⁽⁵⁾Sulcus Time Points: (1) after brushing, (2) 1 hour post rinsing.
⁽⁶⁾Modified by the addition of NaOH.

Apigenin was determined in each toothpaste product and ranged from 0.23 μg/g (Propolis containing toothpaste) to 7964 μg/g (A Modified Acidic Formulation with apigenin salt). In each toothpaste test, the applied amount was ˜1.8 g. With the exception of the chronic alkaline formulation user, no toothpaste subject had a detectable saliva concentration of apigenin at baseline. The sulcus fluid collected immediately after brushing was consistently higher in apigenin than 1 hour later though, importantly, it was still present at this second time point. The apigenin increased markedly immediately after brushing and without rinsing with concentrations ranging from 29.10 to 679.76 μg/ml.

The subject using alkaline formulation with apigenin on a chronic basis had a detectable amount of apigenin (0.72 μg/ml) 12 hours after the last use of the product (i.e., at baseline).

The acidic formulation has a pH 6 and alkaline formulation a pH 10. Salivary apigenin was highest after brushing and 1 hour after rinsing with the modified alkaline apigenin salt but was higher immediately after rinsing with alkaline toothpaste formulation. The various formulations tested demonstrate that different solubilizing technologies can result in therapeutically effective concentrations of bioavailable aglycone flavonoids in the oral cavity both at near, medium and long term time durations. Differing formulations and concentrations can be used to achieve the desired concentrations levels.

### Example 12

### Apigenin Content in Saliva Following the Use of Gum or Lozenge Containing Apigenin Salts

### Primary Objectives:

The primary objective of this study was to evaluate the retention of apigenin in the saliva when using a gum or lozenge of the invention.

### Overall Study Design:

The subjects testing the gum (n=1) and lozenge (n=1) provided saliva samples before and 5 minutes after use of the products but no sulcus fluid was collected. All determinations of apigenin in saliva were conducted in a manner blind to the treatment assignments.

### Sample Preparations:

Gum samples were prepared by infusing a Spearmint Sugarfree Gum by forming a molten mixture of the gum to about 60° C. and adding a few crystals NaOH crystals to achieve a pH of ˜10.0. Apigenin powder was then added to form the soluble sodium salt of Apigenin (˜0.5 wt. %). Additionally, Xylitol ((˜0.5 wt. %) was added to mixture. The molten mixture was then poured into spherical shapes. When cooled, the modified gum was coated with a thin film of Xylitol crystals.

The lozenges were prepared for gummy bears infused with the sodium salt of Apigenin and coated with xylitol crystals. The gummy bears were heated within a microwave oven such that a molten mixture was formed ˜65° C. A concentrated NaOH aqueous concentrate was added to the molten gummy bears to achieve an alkaline pH ˜10.0. Apigenin powder was then added to the mixture to form a 1 wt. % of the soluble sodium Apigenin salt. The molten mixture was the pored into ˜½″ diameter ball shapes. When cooled, the resulting modified gummy bear shapes were coated with a thin layer of xylitol.

### Experimental Methods:

The gum and lozenge samples were pulverized in liquid nitrogen. Apigenin in 0.5 gm of the resulting powder was determined by the same protocol described in Example 11.

### Results

The analytical apigenin concentrations in the gum and lozenge test samples are itemized in Table XV.

TABLE XV
The Analytical Determined Apigenin Content of the Test Samples

| | | | | | Normalized |
|---|---|---|---|---|---|
| | Apigenin | | | Apigenin | Apigenin |
| | Content | Apigenin | | Content | Content to |
| PRODUCT | (mg/ml) | Form | pH | (μg/g) | 1 mg/ml |
| Gum | 0.5 wt. % | Sodium | ~10.0 | 4032 | 8064 |
| (Wrigley's) | | Salt | | | (1 wt. %) |
| Lozenge | 1.0 wt. % | Sodium | ~10.0 | 7963 | 7963 |
| (Gummi Bear) | | Salt | | | (1 wt. %) |

The analytical measured apigenin content in saliva of gum and lozenge as a function of time using gum and lozenge are listed in the Table XVI.

TABLE XVI
The Analytical Determined Apigenin Content
in the Saliva of Gum and Lozenge Subjects

Apigenin Content (μg/ml) in
Saliva Time Points ⁽¹⁾ ⁽²⁾

| TREATMENT | 1 | 2 |
|---|---|---|
| Gum (Wrigley's) | 0.18/(0.36) | 23.36/(46.72) |
| Lozenge (Gummi | 0.09/(0.09) | 75.05/(75.05) |
| Bear) | | |

Note:
⁽¹⁾ Values in parenthesis normalized to 1 wt. % concentration in the gum & lozenge.
⁽²⁾ Saliva Time Points; the subjects testing the gum and lozenge provided saliva samples (1) before and (2) 5 minutes after use.

The use of the gum and lozenge dosage forms was associated with an increase in salivary apigenin after 5 minutes. Low baseline levels of salivary apigenin were noted in the subjects testing the gum and lozenge (0.09 and 0.18 μg/ml, respectively).

### Example 13

### Epigallocatechin Gallate (EGCG) in Saliva Following Product Use

### Primary Objectives:

The primary objective of this study was to evaluate the retention of Epigallocatechin Gallate (EGCG) in saliva following brushing with an alkaline toothpaste containing EGCG.

### Overall Study Design:

A saliva sample with an EGCG containing alkaline toothpaste was obtained (1) before brushing and (2) 1 minute after brushing immediately after rinsing. The subject brushed for 2 minutes using the sulcular brushing technique with ˜1.8 gm toothpaste on a toothbrush.

### Sample Preparations:

A toothpaste sample was prepared by a procedure described in the “Methods of Preparing Formulations of the Invention Section” and Examples 1, 3 & 8. Details of the toothpaste sample are provided in the following Table XVII.

TABLE XVII
Toothpaste Ingredients

| ⁽¹⁾ Toothpaste | Content |
|---|---|
| Ingredients | (wt. %) |
| PeroxiCare ® | 99.8 |
| Ascorbic Acid | 0.1 |
| EGCG | 0.1 |

Note:
⁽¹⁾ A minimal amount of NaOH crystals was added to adjust the formulation to a pH of ~10.5

### Experimental Method:

A 5 ml sample of saliva was collected 10 minutes prior to brushing. Tooth brushing was conducted for 2 minutes with 1.8 gm of toothpaste applied to the brush. One minute after brushing, the oral cavity was rinsed with 50 ml of water. Following rinsing, about 8 ml of saliva was collected. The pH of the saliva was measured. In addition, the saliva samples were further made alkaline with the addition of NaOH crystals and the color of the resulting sample noted.

### Results:

Alkalizing the neutral saliva sample (pH ˜7.0) with sodium hydroxide crystals to a pH ˜10.5 resulted in a red/brown color of the saliva sample indicating that EGCG was retained.

### Example 14

### The Solubility of Various Aglycone Flavonoid Classes in a PS80 Surfactant Solvent

The solubility concentrations of several aglycone flavonoids within acidic formulations are severely limited. For example, all stannous fluoride formulations which reduce gingivitis, plaque, caries etc., are decidedly acidic. Consequently, the thermal processing methods described in U.S. Pat. No. 8,637,569 were used to prepare the PS80/Aglycone Flavonoid concentrates as noted in the Table XVIII. All solubility tests were conducted with 20 ml of PS80 contained within 80 ml Pyrex glass beakers. Complete solubilization of the PS80/Aglycone Flavonoid was achieved at temperature levels exceeding 125° C. The PS80/Aglycone Flavonoid concentrates representing various classes of flavonoids can be added to a variety of oral formulations from pHs preferably ranging 3 to 11 so as to enhance the aglycone flavonoid solubility.

TABLE XVIII
The Solubility of Various Flavonoids Classes
in PS80 via the Thermal Processing Method

| | | | | | PS80 |
|---|---|---|---|---|---|
| | | | ⁽¹⁾ Water | ^{(1) (2)}PS80 | Solution |
| ⁽³⁾FLAVO- | CLASS/ | PURITY | Solubility | Solubility | Temp. |
| NOID | TYPE | (%) | (mg/ml) | (mg/ml) | (° C.) |
| Epigallocate- | Flavanol | 98 | ~25 | 50 | ~150 |
| chin Gallate | | | | | |
| (EGCG) | | | | | |
| Catechin | Flavanol | 90 | ~5 | 50 | ~210 |
| Genistein | Isoflavone | 98 | 0.12 | 50 | ~220 |
| Naringenin | Flavanone | 98 | 0.04 | 50 | ~280 |
| Chrysin | Flavone | 98 | 0.08 | 50 | ~140 |
| Diosmetin | Flavone | 98 | 0.08 | 50 | ~190 |

Note:
⁽¹⁾ Solubility values at 20° C.
⁽²⁾The Flavonoids concentrations are not the maximum solubility levels in the PS80 solvent (Tween 80, High Purity from Croda Inc.)
⁽³⁾The Flavonoids were obtained from Shaanxi Huike Botanical Development Co., Ltd, Xi'an, Shaanxi China

It should be understood that a wide range of changes and modifications could be made to the embodiments described above. It is therefore intended that the foregoing description illustrates rather than limits this invention, and that it is the following claims, including all equivalents, which define this invention.

All references cited in the present specification are hereby incorporated by reference in their respective entireties.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the applied claims.

What is claimed is:

1. A composition comprising:
i) a polyphenol, and
ii) an orally acceptable carrier,
wherein said polyphenol is in the form of a alkali metal salt or a concentrate.

2. A composition as in claim 1 wherein said composition inhibits accumulation of microorganisms upon administration to an oral cavity.

3. A composition as in claim 1 wherein the composition does not include DMSO.

4. A composition as in claim 1 wherein the composition does not include greater that 40% ethanol.

5. A composition as in claim 1, wherein said polyphenol is a flavonoid.

6. A composition as in claim 1, wherein said flavonoid is selected from the group consisting of apigenin, luteolin, kaempferol, quercetin, myricetin, daidzein, genistein, catechins, gallocatechins, naringin, rutin, hesperitin, and a combination of two or more thereof.

7. A composition as in claim 1, wherein said polyphenol is apigenin.

8. A composition as in claim 1, wherein said polyphenol is resveratrol.

9. A composition as in claim 1, wherein said polyphenol is greater than 0.01 percent by weight of the composition.

10. A composition as in claim 1, wherein said polyphenol is 0.1-5 percent by weight of the composition.

11. A composition as in claim 1, wherein the composition has a pH of 5.5-8, and said polyphenol is in the form of a concentrate.

12. A composition as in claim 1, wherein the composition has a pH greater than 8, and said polyphenol is in the form of a alkali metal salt.

13. A composition as in claim 1 having a pH greater than 10.5, and said polyphenol is in the form of a alkali metal salt.

14. A composition as in claim 1 having a pH greater than 10, and said polyphenol is in the form of a alkali metal salt.

15. A composition as in claim 1 wherein said composition is in the form of a liquid, a gel, a paste, a spray, a powder, a gum, a lozenge or a tablet.

16. A composition as in claim 1, further comprising an additive selected from the group consisting of a fluoride compound, cariostatic agent, anti-bacterial agent, anti-tartar agent, anti-inflammatory agent, and a combination of two or more thereof.

17. A composition as in claim 1, further comprising a compound selected from the group consisting of a humectant, abrasive agent, gelling agent, deodorizer, whitening agent, surfactant, binding agent, preservative, coloring agent, buffering agent, stain remover, mineral, vitamin, herb, CoQ10, xylitol, and a combinations of two or more thereof.

18. A composition as in claim 1 further comprising ascorbic acid.

19. A composition as in claim 1, wherein said composition is in the form of a paste or gel and said polyphenol is 0.1-5 percent by weight of the composition.

20. A composition as in claim 1, wherein said composition is in the form of a rinse or spray and said polyphenol is 0.1-5% by weight of the composition.

21. A composition as in claim 1, wherein said composition is in the form of a gum comprising a polyphenol dosage of at least 0.2 mg/stick of gum.

22. A composition as in claim 1, comprising:
i) An alkali metal polyphenol salt,
ii) sodium bicarbonate, and
iii) a peroxide,
wherein said composition is in the form of a toothpaste or gel.

23. A composition as in claim 22 wherein said peroxide is sodium carbonate peroxide.

24. A composition as in claim 23 further comprising a polymer for stabilizing the sodium bicarbonate and/or peroxide.

25. A method of inhibiting the activity of soluble and surface-bound microorganisms responsible for dental caries comprising:
administering to the oral cavity of a mammal a therapeutically effective amount of a composition as in claim 1.

26. A method of providing therapeutically effective sustained levels of a polyphenol in an oral cavity of a mammal comprising:
administering to said oral cavity a composition as in claim 1.

27. A method of delivering a polyphenol systemically to a mammal comprising:
administering buccally to the oral cavity of a mammal a composition as in claim 1.

28. A method for treating an oral disease or condition in a mammal comprising:
administering to the oral cavity of said mammal a therapeutically effective amount of a composition as in claim 1.

29. A method as in claim 28 wherein the oral disease or condition is selected from the group consisting of dental plaque, dental caries, periodontal disease, oral cancer, oral chemotherapy sequelae, gingivitis, herpetic lesions, cold sore, apthous ulcer, toothache, wound, tooth sensitivity, denture stomatitis, fungal, viral or bacterial infection.

30. A method as in claim 28, wherein the administering to the oral cavity comprises administering to one or more of a tooth, a mucosal surface, a tongue surface, a surface on complete or partial dentures, and a combination of two or more thereof.

31. A method as in claim 28, wherein the composition is administered at least once daily.

32. A method as in claim 28, wherein the composition is administered to the oral cavity for a period of about 30-60 seconds and said composition is in the form of a rinse.

33. A method as in claim 28, wherein the composition is administered to the oral cavity for a period of at least 1 minute and said composition in the form of a paste or gel.

34. A method of making a polyphenol containing toothpaste or oral rinse composition comprising:
a) mixing a heat stable polyphenol compound with a heat stable nonionic surfactant to form a mixture,
b) heating said mixture resulting from step a) to a temperature such that said heat stable polyphenol compound is solubilized to form a concentrate,
c) cooling said concentrate resulting from step b),
d) adding the solubilized polyphenol concentrate of step c) to a toothpaste or oral rinse to form said polyphenol containing toothpaste or oral rinse composition.

35. A method as in claim 34 wherein said composition is in a form of a toothpaste.

36. A method as in claim 34 wherein said polyphenol is a flavonoid.

37. A method as in claim 34 wherein said nonionic surfactant is a polysorbate.

38. A method as in claim 34 wherein said mixture is heated to greater than 100° C.

39. A method as in claim 34 wherein said heat stable solubilizing compound is a polysorbate and said flavonoid is apigenin or luteolin.

40. A method of making a polyphenol containing toothpaste or oral rinse composition comprising:
a) mixing a polyphenol with a toothpaste or oral rinse formulation to form a composition,
b) adding an alkali metal hydroxide to the composition of step a) to a pH level of about 10 to form an alkali metal polyphenol salt within said toothpaste or oral rinse formulation, and
c) acidifying the product of step (b) with an acidic agent to form said polyphenol containing toothpaste or oral rinse composition.

41. A method as in claim 40 wherein said polyphenol is a flavonoid.

42. A method as in claim 40 wherein said heat stable solubilizing compound is a polysorbate and said flavonoid is apigenin or luteolin.

43. A method as in claim 40, wherein said flavonoid is selected from the group consisting of kaempferol, quercetin, myricetin, daidzein, genistein, catechins, gallocatechins, naringin, rutin, hesperitin, and combinations of two or more thereof.

44. A method as in claim 40, wherein the alkali metal hydroxide is sodium hydroxide or potassium hydroxide or a mixture thereof.

45. A method as in claim 40, wherein the acidifying agent is citric acid, acetic acid, ascorbic acid, hydrochloric acid or a mixture thereof.
